(19) United States
(12) Patent Application Publication
(10) Pub. No.: US 2024/0389868 A1
(43) Pub. Date: Nov. 28, 2024

(54) AUTOMATIC PRESSURE SENSING IN STRESSFUL SITUATIONS

(71) Applicant: BIOTRONIK SE & Co. KG, Berlin (DE)

(72) Inventors: Christian MOSS, Falkensee (DE); Olaf SKERL, Bad Doberan (DE)

(73) Assignee: BIOTRONIK SE & Co. KG, Berlin (DE)

(21) Appl. No.: 18/691,623

(22) PCT Filed: Sep. 1, 2022
(86) PCT No.: PCT/EP2022/074312

§ 371 Date: Mar. 13, 2024

(30) Foreign Application Priority Data
Sep. 21, 2021 (EP) 21197951.3

(51) Int. Cl. A61B 5/0215; A61B 5/00

(52) U.S. Cl. CPC A61B 5/0215; A61B 5/0031; A61B 2560/0214

(57) ABSTRACT

A mobile communication apparatus for communication with a pressure sensor implanted in a patient. The mobile communication apparatus comprises means for obtaining physical activity data of the patient, and means for sending a wake-up signal to the pressure sensor at least in part based on the physical activity data of the patient.

### CROSS-REFERENCE TO RELATED APPLICATIONS

This application is the United States National Phase under 35 U.S.C. § 371 of PCT International Patent Application No. PCT/EP2022/074312, filed on Sep. 1, 2022, which claims the benefit of European Patent Application No. 21197951.3, filed on Sep. 21, 2021, the disclosures of which are hereby incorporated by reference herein in their entireties.

### TECHNICAL FIELD

The present disclosure generally relates to devices, methods and computer programs for facilitating automatic pressure sensing by implantable pressure sensors in stressful situations. Moreover, the present disclosure relates to a system facilitating such automatic pressure sensing.

### BACKGROUND

Various implantable pressure sensors, such as blood pressure sensors, have been known in the prior art. These may be provided with a battery such that they may enable autarkic operation for a certain period of time, before these may either be explanted or simply be abandoned in the patient's body. They are typically programmed to measure pressure only within certain, e.g., predetermined, periods of time, e.g., during the night-time.

However, it may not always be optimal to only obtain pressure data for such limited periods of time, and it would be desirable to gain more insights into the pressure values of a patient. However, this is generally not possible due to the limited amount of energy that is available to an implanted pressure sensor. It would be conceivable to provide a pressure sensor with a rechargeable battery. However, it is generally difficult to implement reliable and quick charging, and it may not be convenient for the patient to face the risk of quickly running out of battery power, e.g., when the patient is travelling, etc.

There is therefore a need to provide more insight into pressure values of a patient by an implantable pressure sensor without significantly increasing energy consumption.

This need is at least in part met by the aspects disclosed herein.

The present disclosure is directed toward overcoming one or more of the above-mentioned problems, though not necessarily limited to embodiments that do.

### SUMMARY

According to a first aspect, a mobile communication apparatus is provided for communication with a pressure sensor implantable (or implanted) into a patient. The mobile communication apparatus may comprise: means for obtaining physical activity data of the patient; and means for sending a wake-up signal to the pressure sensor at least in part based on the physical activity data of the patient.

At least the above aspect allows, for example, to wake up a pressure sensor depending on a physical activity (that may otherwise be in a passive mode, e.g., having no or only a limited energy consumption, just as for known pressure sensors not making any measurements). This allows targeted measurements of pressure values of a patient if and when the patient undergoes a certain activity, e.g., walking, running, etc., which may be of utmost value for monitoring patients with an existing heart issue (and that may thus have an implanted blood pressure sensor). For example, if the physical activity data of the patient indicates a certain activity of the patient, the (blood) pressure sensor may be woken up, such that it may provide targeted (blood) pressure data at the same time the patient undergoes that activity.

Hence, the insights into the pressure values of a patient may be improved without significantly increasing the energy consumption of the implanted pressure sensor, as the pressure sensor can be woken up in a targeted fashion, e.g., only if a measurement is actually needed or desired. In particular, automatic pressure measurement may be provided in stressful situations (e.g., in which the patient undergoes a certain level of physical activity and/or stress). The stress and/or activity is automatically detected by the mobile communication apparatus and a pressure measurement is automatically triggered by the mobile communication apparatus. The mobile communication apparatus may initiate the wake up of the pressure sensors and may trigger the corresponding measurement accordingly. An interaction of the patient or medical staff may not be needed. The pressure measurement by the pressure sensor is thereby basically controlled and triggered by the mobile communication apparatus.

Moreover, this is enabled in a particularly technically beneficial manner. For example, the mobile communication apparatus may be a (general purpose) portable, handheld and/or wearable device, e.g., a tablet, smartphone, smartwatch, etc. that may be carried and/or worn by most patients anyway, and whose functionality may be enhanced by at least the above aspect. Hence, at least the above aspect may be realized with very limited additional hardware needs.

It is noted that the mobile communication apparatus may be in direct communication with the implantable pressure sensor. However, it may also be in indirect communication with the implantable pressure sensor, e.g., via a further implantable device that may serve as a relay.

In a preferred embodiment of the present invention the mobile communication apparatus is a tablet, a smartphone and/or a smartwatch and the communication apparatus is in direct communication with the implantable pressure sensor. This embodiment would be an easy implementation of the present invention since current tablets, smartphones and/or smartwatches are nowadays equipped with means for obtaining the physical activity.

Tablets, smartphones and/or smartwatches are devices which are used by patients on a daily basis. There is no additional effort needed by the patients for an activity based pressure measurement.

In some examples, the means for obtaining the physical activity data of the patient may comprise an activity sensor. The mobile communication apparatus may thus obtain physical activity data from that sensor which may be first hand, current information. The activity sensor may be internal to the mobile communication apparatus. Thus, physical activity data may be obtained and thus activity may be determined and/or monitored, whenever the mobile communication apparatus is carried and/or worn by the patient. This may provide a significant advantage over a solution in which the activity data is acquired and/or processed by the implantable pressure sensor and/or other implantable devices, since their energy reservoir is typically much more limited than that of (portable or handheld) mobile communication apparatuses (that may comprise a much larger battery that is, in addition, easily rechargeable). In particular, it may allow a continuous monitoring of physical activity which would quickly dry up battery capacities of an implanted device.

For example, the activity sensor may comprise an accelerometer, an inertial sensor, an acceleration sensor, a rotational sensor, an inertial measurement unit (IMU), an inertial navigation system (INS), or a 3D-sensor, etc. An accelerometer may include a piezo sensor, for example. An inertial sensor may include a sensor detecting a rate of rotation, for example.

The mobile communication apparatus may further comprise a processing unit for processing the physical activity data. For example, the processing unit may be implemented by one or more processors, microcontrollers, ASICs, etc. Hence, the processing unit may, for example, process the physical activity data and, based thereon, e.g., if the physical activity data matches certain criteria, trigger the mobile communication apparatus to send the wake-up signal, in particular the mobile communication apparatus may send the wake-up signal directly to the pressure sensor. Hence, a simple and centralized solution for deciding when to wake up the implanted sensor may be provided.

In some examples, the processing unit is adapted to derive at least one activity parameter based on the processing. For example, the activity parameter may relate to an activity index that may be indicative of a level and/or type of motion by a patient. For example, it may indicate an overall acceleration and/or rotation a patient undergoes, e.g., in all three dimensions of space and/or only selected one or more dimensions.

The processing unit may be adapted to compare the at least one activity parameter with a predetermined threshold. For example, to measure pressure values for and above a predetermined stress level of the patient, a corresponding predetermined threshold for the at least one activity parameter may be defined, e.g., an overall acceleration threshold, an acceleration threshold along a certain axis, etc. If the derived activity parameter reaches and/or crosses that threshold, the pressure sensor may be woken up (by a corresponding wake-up signal).

Additionally or alternatively, the processing unit may be adapted to compare the at least one activity parameter with a history associated with the at least one activity parameter. For example, if measurements are made repeatedly, it may be useful that the conditions for triggering a measurement depend on results of a previous measurement. For example, if a first measurement (that may have been made after a predetermined threshold was crossed by the activity parameter) indicates that no (additional) health issues are present. The activity parameter may be compared to an activity parameter value of a first measurement. If that value is reached and/or exceeded, the pressure sensor may be woken up. Additionally or alternatively, the threshold may be increased, e.g., by a predetermined amount, such that a higher level of physical stress may be required to trigger a subsequent measurement by the implantable pressure sensor. In turn, the threshold may be reduced, if it has not been reached for a predetermined period of time.

In a further example, the mobile communication apparatus may further comprise a position sensor, wherein the processing unit is further adapted to process position data obtained from the position sensor. The position sensor may comprise an accelerometer, an orientation sensor, a tilt sensor, etc., for example. For example, the processing may be used to infer a (body) position of the patient, e.g., whether he/she is lying down, sitting, standing, etc.

This may on the one hand improve the determination of a certain activity based on the physical activity data. For example, a certain activity level derived from the data of the activity sensor (e.g., an accelerometer) may be falsely assumed, if the activity, e.g., stems from sitting in a moving car. By means of the position sensor, such false positives may be reduced, e.g., in case the position data indicates that the patient is sitting. Hence, a decision whether or not to wake-up the pressure sensor may be improved.

On the other hand, processing data from the position sensor may allow decisions that would not easily be possible based on data from an activity sensor (alone). For example, the pressure sensor may be woken up in a targeted manner, e.g., if the patient undergoes strong motion (as detected by the activity sensor) while lying down (as detected by the position sensor) which may indicate that the patient has a certain episode while sleeping, or, e.g., if the patient's position is upright and he/she undergoes strong motion which may indicate that the patient is walking or running. Hence, a more targeted assessment of the pressure situation may be enabled.

The mobile communication apparatus may further comprise means for receiving a wake-up response signal from the pressure sensor. This may ensure that the mobile communication apparatus is informed that the wake-up signal has been received by the pressure sensor. For example, the wake-up response signal may be implemented as an acknowledgement signal. For example, the mobile communication apparatus may be configured to re-send the wake-up signal, if no response signal is received within a predetermined time after sending the wake-up signal (e.g., 100 ms, 1 s, 2 s, etc.). This may facilitate using a passive receiver (circuit) in the pressure sensor, that may potentially miss some of the wake-up signals, but at the same time ensure that the precious measurement period (detected based on the physical activity signal) is actually used by timely re-sending the wake-up signal, possibly several times, instead of re-sending only in case no measurement data is received (which typically is sent only after a measurement period of several tens of seconds).

The mobile communication apparatus may further comprise means for receiving first pressure data from the pressure sensor in response to the wake-up signal, in particular via direct communication. Upon reception of the wake-up signal, the pressure sensor may power up and measure first pressure data within a certain measurement period (e.g., for 5 to 50 s, for 10 to 40 s, etc.). The first pressure data may then be sent to the mobile communication apparatus, in particular via direct communication. The first pressure data may thus be acquired by the pressure sensor upon receiving the wake-up signal and thus correspond to current pressure data. In some examples, the wake-up signal may comprise an instruction pertaining to the measurement period, e.g., it may indicate a certain measurement duration (e.g., for 5 to 50 s, for 10 to 40 s, etc.).

The mobile communication apparatus may comprise means for transmitting the first pressure data to a remote communication system. For example, the patient's pressure data may be stored and/or monitored, there. Additionally or alternatively, also the mobile communication apparatus may store and/or monitor the pressure data.

The mobile communication apparatus may further comprise: an internal pressure sensor. The internal pressure sensor may provide second pressure data that reflects an ambient and/or barometric pressure at the patient's location that may then serve to put the first pressure data into perspective and/or to correct the first pressure data.

The mobile communication apparatus may further comprise means for associating the first pressure data with the second pressure data acquired by the internal pressure sensor. For example, first and second pressure data may each comprise one or more time stamps, and they may be associated based on matching or similar time stamps.

Further, the mobile communication apparatus may comprise means for correcting the first pressure data at least in part based on the second pressure data. Hence, corrected pressure data may be provided. For example, the correcting may include a subtraction of first and second pressure data. This may allow removing fluctuations due to barometric pressure fluctuations from the first pressure data. Furthermore the correction would lead to an absolute pressure value at the implantation site.

The mobile communication apparatus may further comprise means for transmitting the first pressure data together with the associated second pressure data and/or the corrected pressure data to the remote communication system. The various pressure data may then be stored and/or monitored, there. It is also possible that the correction as outlined above is done at the remote communication system, e.g., instead of at the mobile communication apparatus.

A second aspect of the present disclosure relates to a method for communication, by a mobile communication apparatus, with a pressure sensor implanted in a patient. The method may comprise: obtaining physical activity data of the patient; and sending a wake-up signal to the pressure sensor at least in part based on the physical activity data of the patient.

It is noted that the method steps as described herein may include all aspects described herein, even if not expressly described as method steps but rather with reference to an apparatus (or device). Moreover, the apparatuses (and devices) as outlined herein may include means for implementing all aspects as outlined herein, even if these may rather be described in the context of method steps.

A third aspect relates to a computer program comprising instructions which when executed by a processor cause a mobile communication apparatus to implement the steps according a method as described herein.

A fourth aspect relates to an implantable pressure sensor. The implantable pressure sensor may comprise: means for receiving a wake-up signal from a mobile communication apparatus, wherein the means for receiving comprises a passive receiver. The implantable sensor thus may be powered off and at the same time always be in a position to be activated, if desired, as decided, e.g., by a mobile communication apparatus that may send the wake-up signal. A passive receiver may comprise an antenna, a transformer and/or a voltage multiplier circuit that may each be passive in that they do not consume any power for being operational. The implantable pressure sensor may be configured as an implantable blood pressure sensor, e.g., for implanting into a blood vessel and/or a heart. For example, the blood pressure sensor may be configured to be implanted into a pulmonary artery.

The implantable pressure sensor may further comprise means for powering up the implantable pressure sensor upon receiving the wake-up signal and for transmitting a wake-up response signal. The wake-up response signal may be sent to the wireless communication apparatus, in particular the wake-up response signal may be sent wireless and directly to the mobile communication apparatus. This may ensure that the mobile communication apparatus is informed that the wake-up signal has been received by the pressure sensor. For example, the wake-up response signal may be implemented as an acknowledgement signal.

Powering up the pressure sensor and transmitting the wake-up response signal upon receiving the wake-up signal may allow to reduce false-positive wake-ups of the sensor: To ensure that the pressure sensor detects the wake-up signal, the detection threshold has to be set such that also relatively weak wake-up signals can be detected, since the wake-up signal may be partially absorbed on its way to the pressure sensor, there may be interference, etc. On the other hand, to avoid that, for example, external noise and/or transmissions of other devices interfering with the passive receiver (circuit) lead to an erroneous detection of a wake-up signal and a subsequent powering-up of the pressure sensor, the threshold should not be set too low: By implementing the wake-up response signal, the mobile communication apparatus is enabled to quickly re-send the wake-up signal, such that the system is operational in a reliable manner even if the detection threshold is set to a relatively high value, since the wake-up signal may be sent repeatedly, until it is detected by the pressure sensor. Hence, due to the relatively high threshold value that may thus be used, “false” wake-ups of the pressure sensor due to noise or signals of other devices may be reduced.

The implantable pressure sensor may further comprise means for acquiring first pressure data in response to receiving the wake-up signal (and possibly after having sent the optional wake-up response signal). The first pressure data may then be sent to the mobile communication apparatus upon completion of the acquisition. The first pressure data may be acquired within a certain measurement period (e.g., for 5 to 50 s, for 10 to 40 s, etc.). In some examples, the wake-up signal may comprise an instruction pertaining to the measurement period, e.g., it may indicate a certain measurement duration (e.g., for 5 to 50 s, for 10 to 40 s, etc.). The implantable pressure sensor may send the first pressure data together with corresponding timing information to the mobile communication apparatus. However, it is also possible that it may be sent without timing information, and, for example, the mobile communication apparatus appends the first pressure data with timing information and/or otherwise attributes timing information to the first pressure data.

A fifth aspect relates to a method that may be carried out by an implantable pressure sensor, as described herein. The method may comprise the following: receiving a wake-up signal from a mobile communication apparatus, wherein the receiving comprises receiving by a passive receiver.

It is outlined that also the method steps as described herein with respect to an implantable pressure sensor may include all aspects described herein, even if not expressly described as method steps but rather with reference to an implantable pressure sensor (as a device).

A sixth aspect relates to a computer program comprising instructions which when executed by a processor cause an implantable pressure sensor to implement the steps according a method as described herein.

A seventh aspect relates to a system comprising a mobile communication apparatus as described herein and an implantable pressure sensor as described herein. The system may facilitate (automatic) intrabody pressure measurement under stress and/or physical activity of the patient.

An eight aspect relates to a method carried out by such a system which may include any of the method steps as described herein with reference to the mobile communication apparatus and the implantable pressure sensor.

Whether described as method steps, computer program and/or means, the functions described herein may be implemented in hardware, software, firmware, and/or combinations thereof. If implemented in software/firmware, the functions may be stored on or transmitted as one or more instructions or code on a computer-readable medium. Computer-readable media include both computer storage media and communication media including any medium that facilitates transfer of a computer program from one place to another. A storage medium may be any available media that can be accessed by a general purpose or special purpose computer. By way of example, and not limitation, such computer-readable storage media can comprise RAM, ROM, EEPROM, FPGA, CD/DVD or other optical disk storage, magnetic disk storage or other magnetic storage devices, or any other medium that can be used to carry or store desired program code in the form of instructions or data structures and that can be accessed by a general-purpose or special-purpose computer, or a general-purpose or special-purpose processor.

Additional features, aspects, objects, advantages, and possible applications of the present disclosure will become apparent from a study of the exemplary embodiments and examples described below, in combination with the Figures and the appended claims.

### BRIEF DESCRIPTION OF THE DRAWINGS

In the following, a brief description of the appended Figures is provided:

FIGS. 1A-B: Schematic representation of an exemplary embodiment of a system according to the present disclosure in communication with a remote communication system.

FIG. 2: Circuit diagram of an exemplary passive receiver circuit of an implantable pressure sensor according to the present disclosure.

### DETAILED DESCRIPTION

In the following, presently preferred exemplary embodiments will be described, mainly with reference to the appended Figures. It is noted that various further embodiments are conceivable.

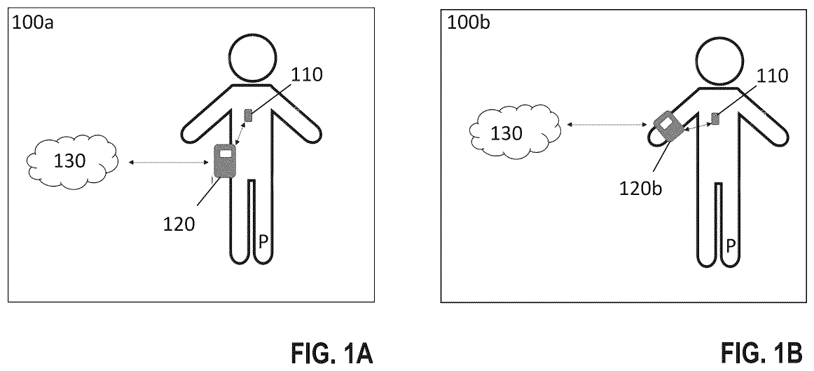

FIGS. 1A and 1B show schematic representations of arrangements 100a and 100b, respectively, of exemplary embodiments of a system according to the present disclosure that is in communication with a remote communication system.

FIG. 1A shows a patient P. Patient P is equipped with a system comprising an implanted pressure sensor 110 and a mobile communication apparatus 120.

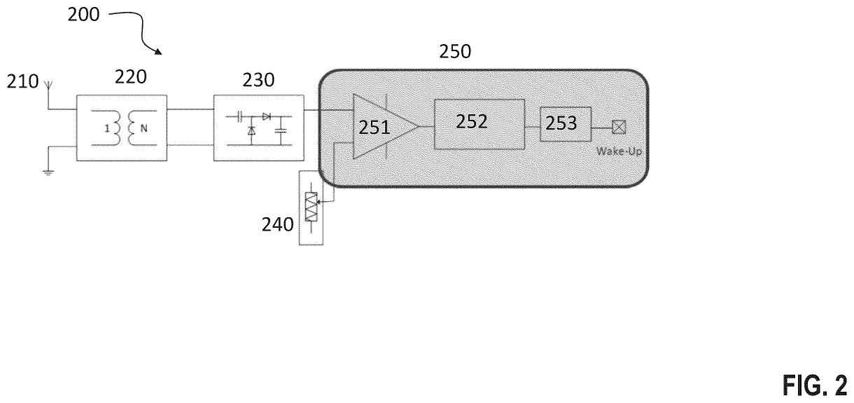

Implanted pressure sensor 110 may comprise a blood pressure sensor. For example, the blood pressure sensor may be implanted into a pulmonary artery of patient P. Pressure sensor 110 may comprise a pressure transducer, a transmitter for sending pressure data to mobile communication apparatus 120, a receiver for receiving data from mobile communication apparatus 120. Moreover, pressure sensor 110 may comprise a passive receiver circuit that may be configured for receiving a wake-up signal from wireless communication apparatus 120. The passive receiver circuit (see exemplary representation in FIG. 2) comprises a passive portion that may be operated without any power supply. The passive receiver circuit may, however, also comprise an active portion. The pressure transducer, transmitter, receiver and the active portion may be powered by a battery of pressure sensor 110. Implanted pressure sensor 110 may generally be in a passive mode in which power supply to the transducer, transmitter and receiver is switched off. Generally, power supply to any element of implanted pressure sensor 110 that may need power (in an active mode) may be switched off in the passive mode, except for the optional active portion of the passive receiver.

Moreover, pressure sensor 110 may comprise at least one anchoring element for anchoring pressure sensor 110 to tissue, for example, tissue of an interior surface of the pulmonary artery.

Mobile communication apparatus 120 may be implemented as a handheld and/or portable device, e.g., a smartphone or a tablet, and may be carried by patient P in a pocket and/or it may be fixable to a belt of patient P, for example. In other embodiments, mobile communication apparatus 120 may be implemented as a smartwatch, smart glasses and/or any other mobile communication device that may be carried by patient P, e.g., in a pocket, in his/her hand, and/or may be fixable to a belt, a wrist, etc., of patient P.

Mobile communication apparatus 120 may be a general purpose device (e.g., a general purpose smartphone, etc.) with its functionality essentially provided by an application (that may e.g., be downloaded from an app store). However, it may, for example, for security reasons, also be provided as a special purpose device, e.g., by the manufacturer of pressure sensor 110.

Mobile communication apparatus 120 may comprise a transmitter to transmit, e.g., a wake-up signal directly to pressure sensor 110. Moreover, it may comprise a receiver to receive, e.g., first pressure data directly from pressure sensor 120. The receiver may use at least one communication technique, e.g., at least one of any type of intrabody communication (IBC), for example, the Medical Implant Communication System (MICS), Bluetooth (Low Energy), Near-Field Communication, etc. The transmitter may use the same or a different one of the listed communication techniques.

Mobile communication apparatus 120 may further comprise an activity and/or position sensor. Signals acquired by the activity and/or position sensor may be provided in a digital form by the sensors and/or digitized by a separate A/D converter of mobile communication apparatus 120. The corresponding activity and/or position data may be processed by a processing unit of mobile communication apparatus 120 that may be implemented, e.g., as outlined herein, e.g., as a processor. Based on the processing, the processing unit may decide whether a wake-up signal is to be sent to pressure sensor 120. For example, such decision (i.e., to send a wake-up signal) may be made, if the activity and/or position data indicates a certain level of stress and/or physical activity of patient P.

For example, the processing unit may derive an activity parameter based on the activity and/or position data. For example, an activity parameter may indicate whether the patient is walking, or running, for example, with a certain speed, etc. It may compare the activity parameter with a predetermined threshold. If the threshold is reached or exceeded, a wake-up signal is sent by mobile communication apparatus 120 to pressure sensor 110. It may also be possible that several activity parameters are determined and compared to corresponding thresholds. Moreover, it is possible to calculate one or more further parameter based on position data provided by the position sensor of mobile communication apparatus 120. For example, a position parameter may indicate whether the patient is lying or in an upright position (or a likelihood therefor).

As an exemplary activity parameter, an activity index may be used. Exemplary algorithms to derive such an activity index will be described in the following. For example, an acceleration component aₓ along a certain axis x may be integrated, e.g., over a certain period of time T, such that the activity index would be ∫_{T} aₓ²(t)dt. For example, the parameter may continuously be evaluated and monitored for exceeding a threshold over a sliding window of time T. Alternatively, the parameter may be evaluated in predetermined intervals, e.g., once every minute, etc. Additionally or alternatively, also an activity index including acceleration along two (e.g., orthogonal) axes x, y may be used (such as ∫_{T} aₓ²(t)+a_{y}²(t)dt), and/or an activity index including acceleration along three (e.g., orthogonal) axes x, y, z (such as ∫_{T} aₓ²(t)+a_{y}²(t)+a_{z}²(t)dt).

It may also be possible to offset the natural acceleration g due to gravitation, when deriving an activity index, such that an activity index as follows may be used, for example:

$${\int }_{T} \vert {a}_{x}^{2} ( t ) + {a}_{y}^{2} ( t ) + {a}_{z}^{2} ( t ) - {g}^{2} \vert dt .$$

The processing unit may be adapted to compare the at least one activity parameter with a predetermined threshold. For example, to measure pressure values for and above a predetermined stress level of the patient, a corresponding predetermined threshold for the at least one activity parameter may be defined, e.g., an overall acceleration threshold, an acceleration threshold along a certain axis, etc. If the derived activity parameter reaches and/or crosses that threshold, the pressure sensor may be woken up (by a corresponding wake-up signal).

Additionally or alternatively to comparing one or more parameters to corresponding threshold(s), the activity and/or position parameters may also be compared to historic values, e.g., a history associated with one or more parameters and/or associated pressure data. For example, the mobile communication apparatus may comprise a storage medium in which past pressure values for certain activity parameters are stored. It may be useful that the conditions for triggering a further pressure measurement depend on one or more results of previous measurements. For example, if a first measurement (that may have been made after a predetermined threshold was crossed by the activity parameter) indicates that no (additional) health issues are present (and/or that the pressure values were within a predetermined range), the threshold may be increased, e.g., by a predetermined amount, such that a higher level of physical stress may be required to trigger a subsequent measurement by the implantable pressure sensor. On the other hand, if the parameter derived for a patient has not exceeded a predetermined threshold for a predetermined time, e.g., one day, one week or one month, etc., the threshold may be reduced to the maximum parameter value derived during the last day, week or month, respectively. A missing threshold crossing may indicate a reduced activity by the patient, which may require a corresponding adjustment of the threshold.

Additionally or alternatively, also position data may be processed, e.g., to infer a position of the patient, e.g., whether he/she is lying down, sitting, standing, etc., e.g., as outlined herein.

Various algorithms may be provided to mobile communication apparatus 120 to derive activity and/or position parameters, and/or to derive a decision whether to wake-up the pressure sensor 110, based thereon, e.g., in the form of a corresponding computer program (e.g., an application).

While the mobile communication apparatus 120 processes the data, pressure sensor 110 is usually in a passive state or passive mode, with possibly essentially all power consuming parts of pressure sensor 110 turned off (except for the optional active portion of the passive receiver circuit, cf. FIG. 2). Once it receives the wake-up signal (the wake-up signal may be a high-frequency electromagnetic signal) from mobile communication apparatus 120, pressure sensor 110 may be woken up, and, e.g., the transmitter of pressure sensor 110 may be powered up to transmit a wake-up response signal to mobile communication apparatus 120. Moreover, the transducer of pressure sensor 110 may be powered up, and pressure data may be acquired. The pressure data may then be transmitted to the mobile communication apparatus 120.

Mobile communication apparatus 120 may be in communication with a remote communication system 130. Remote communication system 130 may be implemented as a server based system and/or cloud-based system. Mobile communication apparatus 120 may communicate the (first) pressure data received from pressure sensor 110 to remote communication system 130. Remote communication system 130 may store and/or monitor and/or process the first pressure data. If needed, it may optionally return data to mobile communication system 120, e.g., to adjust one or more aspects of the algorithms described herein that are carried out on mobile communication system 120 to decide whether to send a wake-up signal. Additionally or alternatively, also a measurement time (optionally) indicated in a wake-up signal may be adjusted. For communication with remote communication system 130, mobile communication system 120 may comprise a wireless interface, e.g., based on WiFi, 3G, 4G and/or 5G, etc., for example, to communicate over the internet.

Remote communication system 130 may provide the received data and/or analysis results based thereon to persons having access to it, such as a doctor, hospital, relatives, etc. taking care of patient P.

Mobile communication apparatus 120 may also comprise an internal pressure sensor such that second pressure data (pertaining to barometric and/or ambient pressure) may be provided. For example, whenever first pressure data is received by mobile communication apparatus 120, it may acquire a pressure value indicating the barometric/ambient pressure as provided by the internal pressure sensor. Alternatively, for the period of time during which pressure sensor 110 is instructed to acquire first pressure data, mobile communication apparatus 120 may acquire corresponding second pressure data. It may also be possible that the second pressure data is acquired continuously or at least regularly regardless of whether first pressure data is currently being acquired. Mobile communication apparatus 120 may correct the first pressure data based on the (corresponding) second pressure data to obtain corrected pressure data. Additionally or alternatively to the first pressure data, mobile communication apparatus 120 may send the corrected pressure data or the second pressure data to the remote communication unit 130. In other examples, the mobile communication apparatus 120 may not correct the first pressure data, but, e.g., send first pressure data with corresponding second pressure data to remote communication system 130.

In an example, mobile communication system 120 may forward the first pressure data, the second pressure data and activity data (used to trigger the acquisition of the first pressure data, and/or additionally acquired during the time the first pressure is acquired (the latter may allow a double-check whether the first pressure data indeed relates to the desired level of activity)) to remote communication system 130.

FIG. 1B shows patient P which is equipped with a system comprising an implanted pressure sensor 110 and a mobile communication apparatus 120b. The system may be in communication with a remote communication system 130. Pressure sensor 110 and remote communication system 130 may be essentially identical to those as described with reference to FIG. 1A. Also mobile communication apparatus 120b may generally be similar to mobile communication apparatus 120 as described with reference to FIG. 1A. However, mobile communication apparatus 120b may specifically be configured to be worn around a wrist of patient P, e.g., it may be implemented as a watch, such as a smartwatch.

FIG. 2 shows an exemplary passive receiver circuit 200 of pressure sensor 110 adapted to receive a wake-up signal as described herein. It is noted that the transmitter of pressure sensor 110 (that may send the wake-up response signal) and the receiver of pressure sensor 110 that may be provided to receive further data from mobile communication apparatus 120 may, at least in part, share one or more elements with passive receiver circuit 200 (in which case the latter may still be a separate logical entity). Alternatively, passive receiver circuit 200 may be a separate physical entity.

Passive receiver circuit 200 may comprise an antenna 210. Antenna 210 may be coupled to a transformer 220. Transformer 220 may comprise a piezoelectric MEMS transformer. Transformer 220 may be coupled to a passive voltage multiplier circuit 230 (that may also comprise an envelope detector). Transformer 220 may adjust the signal received from antenna 210 such that it can be amplified by voltage multiplier circuit 230. To avoid unwanted activation by noise, the output of voltage multiplier circuit 230 is coupled to an input of a comparator 251. A bias 240 is coupled to a further input of comparator 251.

Antenna 210, transformer 220, voltage multiplier circuit 230 and bias 240 may be considered to form the passive portion of passive receiver circuit 200. These elements may essentially not consume any power. Hence, without consuming power, they may virtually permanently be active to receive and adjust wake-up signals.

If a wake-up signal is received that leads to a signal at comparator 251 that exceeds that of bias 240, it is coupled to preamble detector 252. Preamble detector 252 may detect a preamble of the wake-up signal in order to perform a plausibility check, whether the signal exceeding that of bias 240 indeed originates from a wake-up signal. Wake-up signal may comprise or essentially consist of a burst signal that includes a preamble, identifying the wake-up signal as a wake-up signal. In an example, no further data may be comprised by the wake-up signal.

If the plausibility check is successful, the wake-up signal (and/or any representation thereof, e.g., a bit) may be written into a latch 253, which may then trigger an internal wake-up call, triggering the pressure sensor 110 to implement the steps outlined herein. In particular, a transmitter of pressure sensor 110 may be powered up to send a wake-up response signal to mobile communication apparatus 120, wherein the transmitter may be part of a radio interface via which pressure sensor 110 and mobile communication apparatus 120 usually communicate.

After the method steps have been carried out, pressure sensor 110 may again power down. Essentially all elements of pressure sensor 110 may be powered down, except for active portion 250 of passive receiver circuit that comprises comparator 251, preamble detector 252 and latch 253. However, the active portion 250 may be designed to be operated with very low currents, such as up to or below 100 nA, e.g., 5 to 100 nA, preferably up to or below 25 nA, such as 5 to 40 nA or 25 to 40 nA.

It will be apparent to those skilled in the art that numerous modifications and variations of the described examples and embodiments are possible in light of the above teachings of the disclosure. The disclosed examples and embodiments are presented for purposes of illustration only. Other alternate embodiments may include some or all of the features disclosed herein. Therefore, it is the intent to cover all such modifications and alternate embodiments as may come within the true scope of this invention, which is to be given the full breadth thereof. Additionally, the disclosure of a range of values is a disclosure of every numerical value within that range, including the end points.

1. Mobile communication apparatus for communication with a pressure sensor implantable into a patient, comprising:
means for obtaining physical activity data of the patient;
means for sending a wake-up signal to the pressure sensor at least in part based on the physical activity data of the patient.

2. Mobile communication apparatus according to claim 1,
wherein the mobile communication apparatus is a tablet, a smartphone and/or a smartwatch and the communication apparatus is in direct communication with the implantable pressure sensor.

3. Mobile communication apparatus according to claim 1, wherein the means for obtaining comprises an activity sensor.

4. Mobile communication apparatus according to claim 1, further comprising a processing unit for processing the physical activity data.

5. Mobile communication apparatus according to claim 4, wherein the processing unit is adapted to derive at least one activity parameter based on the processing.

6. Mobile communication apparatus according to claim 5, wherein the processing unit is adapted to compare the at least one activity parameter with a predetermined threshold and/or with a history associated with the at least one activity parameter.

7. Mobile communication apparatus according to claim 4, further comprising a position sensor, wherein the processing unit is further adapted to process position data obtained from the position sensor.

8. Mobile communication apparatus according to claim 1, further comprising means for receiving a wake-up response signal from the pressure sensor.

9. Mobile communication apparatus according to claim 1, further comprising means for receiving first pressure data from the pressure sensor in response to the wake-up signal, the first pressure data acquired by the pressure sensor upon receiving the wake-up signal.

10. Mobile communication apparatus according to claim 9, further comprising:
an internal pressure sensor; and
means for associating the first pressure data with second pressure data acquired by the internal pressure sensor.

11. Mobile communication apparatus according to claim 10,
further comprising:
means for correcting the first pressure data at least in part based on the second pressure data; and/or
means for transmitting the first pressure data together with the associated second pressure data to a remote communication system.

12. Method for communication, by a mobile communication apparatus, with a pressure sensor implanted in a patient, comprising:
obtaining physical activity data of the patient;
sending a wake-up signal to the pressure sensor at least in part based on the physical activity data of the patient.

13. Computer program comprising instructions which when executed by a processor cause a mobile communication apparatus to implement the steps according to the method of claim 11.

14. Implantable pressure sensor, comprising:
means for receiving a wake-up signal from a mobile communication apparatus;
wherein the means for receiving comprises a passive receiver.

15. Implantable pressure sensor according to claim 13, further comprising:
means for powering up the implantable pressure sensor upon receiving the wake-up signal; and
transmitting a wake-up response signal.

16. A system comprising a mobile communication apparatus according to claim 1 and an implantable pressure sensor comprising means for receiving a wake-up signal from a mobile communication apparatus, wherein the means for receiving comprises a passive receiver.
